(19) United States
(12) Patent Application Publication
(10) Pub. No.: US 2008/0188755 A1
(43) Pub. Date: Aug. 7, 2008

(54) Ultrasound Transducer Assembly Having Improved Thermal Management

(75) Inventor: Jeffrey Hart, Port Royal, PA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/912,617

(22) PCT Filed: Apr. 20, 2006
(86) PCT No.: PCT/IB2006/051228

§ 371 Date: Oct. 25, 2007

Related U.S. Application Data
(60) Provisional application No. 60/674,494, filed on Apr. 25, 2005.

(51) Int. Cl. A61B 8/13

(52) U.S. Cl. 600/459

(57) ABSTRACT

An improved thermal management of an ultrasound transducer assembly is provided. The ultrasound transducer assembly includes an ultrasound transducer operable to transmit ultrasound energy along a propagation path; and a self-contained cooling system thermally coupling the ultrasound transducer to at least one heat sink. The self-contained cooling system includes at least one heat transfer member. The self-contained cooling system defines a heat flow from the ultrasound transducer assembly to the heat sink via the at least one heat transfer member. The propagation path of the ultrasound energy is opposite in direction to the heat flow path. The heat transfer process is augmented by the addition of a thermoelectric cooler positioned in thermal communication with the ultrasound transducer assembly. The self-contained cooling system provides for minimum thermal resistance, while the thermoelectric cooler maintains the heat flow in a positive direction and maintains positive thermal gradients thus enhancing the heat flow to the heat sink.

The present disclosure relates generally to medical ultrasound imaging systems for visualizing soft tissue organs in the interior regions of the body. More particularly, the present disclosure relates to an ultrasound transducer assembly having improved thermal management.

Ultrasound imaging is a medical diagnostic imaging which permits the visualization of soft tissue organs in the interior regions of the body. An ultrasound imaging process generally involves placing an ultrasound transducer assembly or transducer probe against the skin of a patient near the region of interest, such as, for example, against the back to image the kidneys.

The ultrasound transducer assembly is operable to transmit ultrasound energy along a propagation path and includes a transducer array and corresponding electrical circuitry in operative communication with the transducer array. Despite its success and overall acceptance as a preferred technique for non-invasively imaging a number of soft tissue organs, the design of an ultrasound transducer assembly presents a number of challenges. In particular, ultrasound transducer assembly requires a thermal management system in order to limit the surface temperature of the ultrasound transducer assembly by managing the heat generated by the transducer array and corresponding electrical circuitry. In addition, there are regulatory and safety requirements that must be satisfied in order to sustain optimal performance of the ultrasound transducer assembly. For example, it is desirable that the housing of the ultrasound transducer assembly be comfortably cool to prevent excess perspiration in the hand of the operator.

Moreover, as new innovations in the design of ultrasound transducer assemblies are developed, such as, for example, microbeam forming technology, it is increasingly important to incorporate an effective and economical thermal management system in the ultrasound transducer assembly in order to ensure proper functioning of the ultrasound transducer assembly.

To address these concerns, thermal management of ultrasound transducer assemblies has long been an important issue in the design of ultrasound transducer assemblies. There is significant prior art describing various methods to transport heat energy generated by the ultrasound transducer assembly elements. For example, one method makes use of passive cooling mechanisms wherein the heat energy generated by the ultrasound transducer housed by the ultrasound transducer assembly is passively dissipated to a heat sink usually, the cable and/or the housing. However, passive cooling mechanisms can be ineffective in removing heat energy from multiple, localized regions of the ultrasound transducer assembly. A second method incorporates active cooling mechanisms generally in fluid communication with external cooling fluids. An active cooling mechanism incorporates fans, suction devices, pumps, and/or other energy consuming means to dissipate heat from the ultrasound transducer assembly. Active cooling systems are expensive and include elaborate cooling devices. Examples of active cooling mechanisms are described in U.S. Pat. No. 5,560,362 issued to Sliwa Jr., et al.

The present disclosure obviates the disadvantages of the prior art by providing an ultrasound transducer assembly having a self-contained cooling system thermally coupling multiple heat sources in the ultrasound transducer to a heat sink. The ultrasound transducer assembly further includes a thermoelectric cooler thermally coupled to the ultrasound transducer for augmenting the heat transfer process.

The present disclosure provides improved thermal management of an ultrasound transducer assembly. In particular, the present disclosure provides an ultrasound transducer assembly adapted to effectively manage the thermal energy it generates. The ultrasound transducer assembly of the present disclosure includes an ultrasound transducer operable to transmit ultrasound energy along a propagation path. The ultrasound transducer includes a transducer array and corresponding electric circuitry in operable communication with the transducer array; and a cooling system thermally coupling at least one of the transducer array and the corresponding electrical circuitry to at least one heat sink. The cooling system defines a low resistance heat flow path from the sources within the transducer to the sink(s) and maintains the direction of heat flow in a direction substantially opposite the propagation path of the ultrasound energy.

In one aspect of the presently disclosed ultrasound transducer assembly, the heat transfer process is augmented by the addition of a thermoelectric cooler positioned in thermal communication with the ultrasound transducer assembly. More in particular, the thermoelectric cooler is thermally coupled with the corresponding electrical circuitry. The thermoelectric cooler is activated when the temperature of the electrical circuitry is higher than the temperature of the transducer array which would cause heat to propagate toward the patient applied surface. The thermoelectric cooler is adapted to bias the temperature of the corresponding electrical circuitry lower than the transducer array temperature to prevent heat conduction from the electrical circuitry toward the transducer array. Thus, the self-contained cooling system provides for minimum thermal resistance while the thermoelectric cooler maintains the heat flow in the positive direction (towards one or more heat sinks) by maintaining a positive thermal gradient between the array and the heat sink.

Preferably, in an alternative embodiment, the transducer array and the corresponding electrical circuitry may be combined into one integral assembly. Thus, the thermal load generated by the transducer array and the corresponding electrical circuitry are combined into a compact space. The self-contained cooling system thermally couples these combined loads to the at least one heat sink.

The ultrasound transducer assembly of the present disclosure further includes a housing, and a cable assembly for connecting the ultrasound transducer assembly to an imaging station. The thermal conductivity of the housing may be enhanced by material selection, i.e. the housing is constructed of a thermally conductive material, such as, for example, loaded-thermally conductive polymer and/or metal. Alternatively, the thermal conductivity of the housing may be increased by internal metallization of a traditional unfilled polymer. In a preferred embodiment, the at least one heat sink may be the housing and/or the cable assembly.

A method of dissipating thermal energy generated by an ultrasound transducer assembly is also envisioned. The method includes the steps of providing a self-contained cooling system within an ultrasound transducer assembly thermally coupling at least one of an ultrasound transducer array and corresponding electrical circuitry of the ultrasound transducer array to at least one heat sink. The self-contained coolant system includes at least one heat transfer member partially filled with a working fluid and defines a heat flow path from at least the ultrasound transducer array and the corresponding electrical circuitry to the at least one heat sink via the at least one heat transfer member. The method further includes enabling the thermal energy to propagate along the heat flow path during operation of the ultrasound transducer assembly, such that the heat flow path propagates the thermal energy in a direction opposite an ultrasound propagation path of the ultrasound transducer assembly. The method further includes the step of providing a thermoelectric cooler thermally coupled with the corresponding electrical circuitry of the ultrasound transducer array in order to maintain heat flow in a direction substantially opposite the propagation of ultrasound energy.

Other features and advantages of the present disclosure will become apparent from the following detailed description, taken in conjunction with the accompanying drawings, which illustrate, by way of example, the principals of the invention.

The foregoing features of the present disclosure will become more readily apparent and will be better understood by referring to the following detailed description of preferred embodiments, which are described hereinbelow with reference to the drawings wherein:

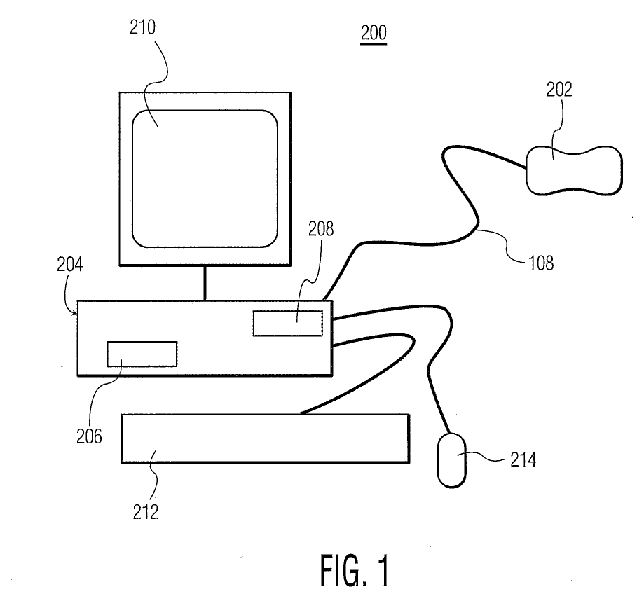

FIG. 1 is a perspective view of a medical ultrasound diagnostic imaging system in accordance with the principles of the present disclosure;

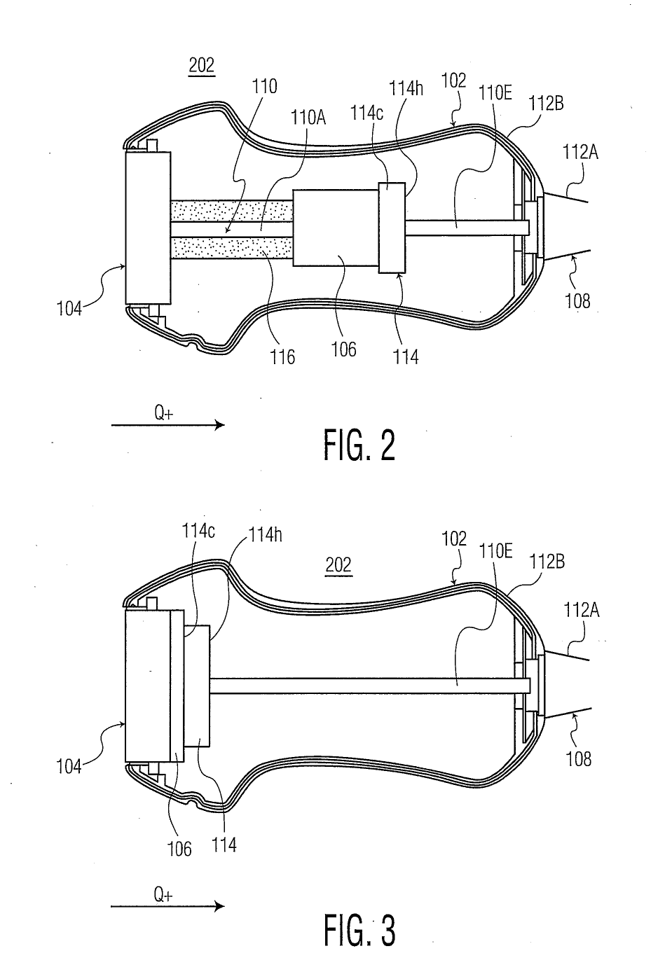

FIG. 2 is partial cross-sectional view of an ultrasound transducer assembly illustrating the self-contained cooling system in accordance with the present disclosure; and

FIG. 3 is partial cross-sectional view of an alternative embodiment of an ultrasound transducer assembly illustrating the self-contained cooling system in accordance with the present disclosure.

The medical ultrasound imaging system of the present disclosure provides an ultrasound transducer assembly having improved thermal management. The ultrasound transducer assembly includes an ultrasound transducer array and corresponding electrical circuitry and is adapted for transmitting ultrasound energy along a propagation path. Moreover, the ultrasound transducer assembly of the present disclosure is capable of conducting heat from all heat sources within the assembly, i.e. ultrasound transducer array and corresponding electrical circuitry, to at least one heat sink.

Referring now in detail to the drawing figures, in which like reference numerals identify similar or identical elements, a medical ultrasound imaging system in accordance with the present disclosure is illustrated, and is designated generally as ultrasound imaging system 200. In the following description, as is traditional, the term “proximal” refers to the portion of the instrument closest to the operator, while the term “distal” refers to the portion of the instrument remote from the operator.

Referring initially to FIG. 1, there is illustrated a medical ultrasound diagnostic imaging system 200 constructed in accordance with the principles of the present disclosure. Ultrasound imaging system 200 is particularly adapted for use in medical diagnostic imaging techniques. Generally, ultrasound imaging system 200 includes two principal subassemblies, namely, imaging workstation 204 and ultrasound transducer assembly 202 connected to imaging workstation 204. Ultrasound imaging system 200 has the objective of providing an ultrasound transducer assembly 202 having a self-contained cooling system adapted to conduct heat from ultrasound transducer assembly 202 to at least one heat sink. In particular, ultrasound imaging system 200 provides an improved thermal management system for ultrasound transducer assembly 202 by thermal transport of heat or thermal energy from the ultrasound transducer 202 to at least one heat sink.

With continued reference to FIG. 1, imaging workstation 204 may be any imaging workstation suitable for use in medical ultrasonography. In one preferred embodiment, imaging workstation 204 includes at least one processor 206 for performing calculations and at least one storage device 208, such as, for example, a hard drive, RAM disk, etc., for temporary or long term storage of image data acquired by the ultrasound transducer assembly 202. Imaging workstation 204 further provides video display 210 for displaying the image data, and input devices such as keyboard 212 and mouse 214.

With reference now to FIGS. 2-3, ultrasound transducer assembly 202 will now be discussed. Ultrasound transducer assembly 202 preferably includes an ultrasound transducer operable to transmit ultrasound energy along a propagation path and having an ultrasound transducer array and corresponding electrical circuitry in operative communication with the ultrasound transducer array. Ultrasound transducer assembly 202 further includes housing 102, transducer array 104, corresponding electrical circuitry 106 in operative communication with transducer array 104, and cable assembly 108. Cable assembly 108 is preferably a flexible coaxial cable for connecting ultrasound transducer assembly 202 to imaging workstation 204. The transducer array 104 and corresponding electrical circuitry 106 are preferably connected through hard wired communication, however, it is envisioned that the connection may be wireless or a combination of hard wired and wireless connections.

Ultrasound transducer assembly 202 further includes a self-contained cooling system 110 thermally coupling the transducer array 104 and corresponding electrical circuitry 106 to heat sink 112. The primary function of self-contained cooling system 110 is the thermal management of multiple heat sources in ultrasound transducer 202, i.e. transducer array 104 and corresponding electrical circuitry 106. Alternatively, self contained cooling system 110 thermally couples one of transducer array 104 or corresponding electrical circuitry 106 to heat sink 112. Self-contained cooling system 110 conducts heat from transducer array 104 and corresponding electrical circuitry 106 to heat sink 112. Self-contained cooling system 110 defines a heat flow path (depicted by directional arrow “Q+”). The propagation path of the ultrasound energy generated by ultrasound transducer assembly 202 is opposite in direction to the heat flow path defined by self-contained cooling system 110. Preferably, the components of the self-contained cooling system 110 include materials with large thermal conductivity, i.e. low thermal resistance, such as, for example, copper.

With continued reference to FIG. 2, the primary components of the self-contained cooling system 110 are first and second heat transfer members 110A and 110E. First heat transfer member 110A can be partially filled with a working fluid to thermally couple transducer array 104 to electrical circuitry 106 or to a heat sink 112. Second heat transfer member 110E can be partially filled with a working fluid to thermally couple corresponding electrical circuitry 106 to one or more heat sinks 112A and 112B. Heat sink 112A includes cable assembly 108 and heat sink 112B includes the thermally conductive housing 102. Heat is dissipated by thermally coupling heat transfer member 110E to heat sink 112A by extending a proximal end of second heat transfer member 110E into heat sink 112A via cable assembly 108. Alternatively, heat may be dissipated by thermally coupling heat transfer member 110E to heat sink 112B via potting with a thermally conductive material.

The thermal conductivity of the housing 102 may be enhanced by material selection, i.e. the housing is constructed of a thermally conductive material, such as, for example, loaded-thermally conductive polymer and/or metal. Alternatively, the effective thermal conductivity of the housing 102 may be increased by internal metallization of a traditional unfilled polymer.

Thermoelectric cooler 114 may be included in order to augment the heat transfer process of self-contained cooling system 110. Thermoelectric cooler 114 is thermally coupled in the cooling system between the source(s) and the sink(s). Thermoelectric cooler 114 may be any thermoelectric cooler having a closed DC circuit and suitable for use in applications where temperature cooling is desired. As shown in the figures, thermoelectric cooler 114 includes a hot surface 114h and a cold surface 114c. Cold surface 114c is thermally coupled to a heat source such as, for example, electrical circuitry 106. Hot surface 114h is thermally coupled to heat sink 112. In the embodiment shown in FIG. 2, thermoelectric cooler 114 is thermally coupled to the electrical circuitry 106. Hot surface 114h of thermoelectric cooler 114 is then coupled to heat sink 112A via second heat transfer member 110E of self-contained cooling system 110. Thermoelectric cooler 114 maintains a positive thermal gradient. That is, thermoelectric cooler 114 maintains the heat flow emanating from transducer array 104 and electrical circuitry 106 in the positive direction, depicted by directional arrow “Q+”, i.e., towards heat sink 112A.

Thermoelectric cooler 114 is activated when the temperature of the electrical circuitry 106 is higher than the temperature of the transducer array 104. In addition, other criteria such as array temperature and imaging mode may be used to activate the active cooling system. Thus, thermoelectric cooler 114 will bias the temperature of the electrical circuitry 106 lower than the temperature of transducer array 104 to prevent heat flow from the electrical circuitry to the array structure, i.e., in a direction opposite the direction shown by directional arrow “Q+”.

With particular reference to FIG. 3, an alternative embodiment is illustrated. The embodiment illustrated in FIG. 3 is similar to that of FIG. 2, except that the electrical circuitry 106 is integrally located in the array placing the thermal sources in close proximity and the first heat transfer member 110A is removed. Self-contained cooling system 110 thermally couples the combined thermal loads to heat sink 112A and or 112B. The active cooling system can then be used as previously described to augment heat flow to the sinks 112A and or 112B.

It will be understood that various modifications and changes in form and detail may be made to the embodiments of the present disclosure without departing from the spirit and scope of the invention. Therefore, the above description should not be construed as limiting the invention but merely as exemplifications of preferred embodiments thereof. Those skilled in the art will envision other modifications within the scope and spirit of the present invention as defined by the claims appended hereto. Having thus described the invention with the details and particularity required by the patent laws, what is claimed and desired protected is set forth in the appended claims.

1. An ultrasound transducer assembly comprising:
an ultrasound transducer operable to transmit ultrasound energy along a propagation path, said ultrasound transducer comprising a transducer array and corresponding electrical circuitry in operative communication with said transducer array; and
a self-contained cooling system thermally coupling at least one of said transducer array and said corresponding electrical circuitry to at least one heat sink, said self-contained cooling system including at least one heat transfer member, wherein the self-contained cooling system defines a heat flow path from at least one of the transducer array and corresponding electrical circuitry to the at least one heat sink via the at least one heat transfer member, said propagation path of said ultrasound energy is substantially opposite in direction to said heat flow path.

2. The ultrasound transducer of claim 1, further comprising a thermoelectric cooler thermally coupled with at least one source, transducer array 104 or electrical circuitry 106.

3. The ultrasound transducer of claim 1, wherein said at least one heat transfer member includes a first element positioned between said transducer array and said corresponding electrical circuitry, and a second element positioned between the corresponding electrical circuitry and the at least one heat sink.

4. The ultrasound transducer of claim 1, wherein a central axis of the at least one heat transfer member is substantially aligned with a central axis of the at least one heat sink.

5. The ultrasound transducer of claim 1, wherein the at least one heat sink includes at least a portion of a cable assembly.

6. The ultrasound transducer of claim 1, further comprising a housing encasing said self-contained cooling system, wherein the at least one heat sink is the housing.

7. The ultrasound transducer of claim 6, wherein the at least one heat sink includes the housing and a cable assembly.

8. The ultrasound transducer of claim 1, wherein the at least one heat transfer member is partially filled with said working fluid.

9. The ultrasound transducer of claim 1, wherein the at least one heat transfer member is thermally coupled to the transducer array and extends through a portion of said at least one heat sink.

10. The ultrasound transducer of claim 1, wherein said transducer array is located in close proximity to said corresponding electrical circuitry.

11. The ultrasound transducer of claim 1, wherein the at least one heat sink is constructed from a thermally conductive polymer.

12. The ultrasound transducer of claim 1, wherein the cooling fluid includes a combination of liquid and gas phases.

13. An ultrasound transducer assembly comprising:
at least one thermally conductive heat sink;
a transducer mounted in operative communication with the at least one thermally conductive heat sink, the transducer operable to transmit ultrasound energy along a propagation path, said transducer comprising a transducer array and corresponding electrical circuitry coupled to said transducer array;
a self-contained cooling system in thermal communication with the transducer for conducting heat generated by the transducer array and corresponding electrical circuitry to said at least one heat sink, wherein said self-contained cooling system defines a heat flow from the transducer array and corresponding electrical circuitry to said at least one heat sink via at least one heat transfer member, wherein said propagation path and the heat flow being in opposite directional path.

14. The ultrasound transducer of claim 13, further comprising a thermoelectric cooler thermally coupled with said corresponding transducer array 104 or electrical circuitry 106.

15. The ultrasound transducer of claim 13, wherein the thermoelectric cooler is mounted adjacent to the electrical circuitry.

16. The ultrasound transducer of claim 13, wherein the self-contained cooling element extends into the at least one heat sink.

17. The ultrasound transducer of claim 13, wherein the at least one heat transfer member is partially filled with said working fluid.

18. The ultrasound transducer of claim 13, wherein the at least one heat sink is constructed from a thermally conductive material, said thermally conductive material is selected from a group consisting of thermally conductive polymer and metal.

19. A method of dissipating thermal energy generated by an ultrasound transducer assembly, comprising the steps of:
providing an ultrasound transducer assembly; and
providing a self-contained cooling system within said ultrasound transducer assembly thermally coupling at least one of an ultrasound transducer array and corresponding electrical circuitry of said ultrasound transducer array to at least one heat sink, said self-contained cooling system including at least one heat transfer member filled with a working fluid, defining a heat flow path of heat from at least one of the transducer array and corresponding electrical circuitry to the at least one heat sink via the at least one reservoir, and
enabling said thermal energy to propagate along said heat flow path during operation of said ultrasound transducer assembly, wherein said heat flow path propagates said thermal energy in a direction opposite an ultrasound propagation path of said ultrasound transducer assembly.

20. The method of claim 19, further comprising the step of providing a thermoelectric cooler thermally coupled with said ultrasound transducer.
